(19) United States
(12) Patent Application Publication
(10) Pub. No.: US 2010/0098738 A1
(43) Pub. Date: Apr. 22, 2010

(54) SURFACE BOUND ACTIVES

(75) Inventor: Richard Milner, Selby (GB)

Correspondence Address: DIANA HOUSTON;SMITH & NEPHEW, INC., 1450 BROOKS ROAD, MEMPHIS, TN, 38116, US

(73) Assignee: SMITH & NEPHEW, INC., Memphis, TN (US)

(21) Appl. No.: 12/526,457

(22) PCT Filed: Feb. 11, 2008
(86) PCT No.: PCT/GB2008/000469

§ 371 Date: Aug. 7, 2009

(30) Foreign Application Priority Data
Feb. 12, 2007 (GB) 0602665.1

(51) Int. Cl. A61F 2/00; A61K 38/16

(52) U.S. Cl. 424/423; 514/8

(57) ABSTRACT

An implantable metal device having an active biosurface, the device comprising a non-peptide polymer spacer having a plurality of target sites, at least one or more of the target sites binding a biologically active agent, wherein the target sites are derived from a plurality of carbonyl, epoxy, hydroxyl or thiol target reactive groups or a combination thereof or wherein the biologically active agent is any agent which is capable of modifying the behaviour of cells; a process for generating an active biosurface at the surface of the implantable metal device, comprising contacting a non-peptide polymer spacer having a plurality of target reactive groups with a biologically active agent having one or more reactive functional groups and reaction thereof, thereby binding the biologically active agent to the polymer wherein the, polymer is anchored to the surface of the implantable metal device or subsequently anchoring the polymer to the surface of the implantable metal device by means of unreacted target reactive groups; the implantable metal device having a surface which is receptive for tethering the biologically active agent; a chemical intermediate comprising the polymer spacer anchored to the chemical anchor; processes for the preparation thereof; a method of treating an animal comprising inserting the implantable metal device into a site in need thereof on said animal; and uses thereof as an implantable or subcutaneous metal device, or non-surgical device or a coating thereof, preferably any orthopaedic, cardiovascular, circulatory system or dental implant, tissue engineering device, fixation device, reconstructive device or joint member or trauma-related devices or research tools or particles for shaping into such form.

This invention relates to implantable metal devices having an active biosurface comprising surface bound biologically active agents, particularly to implantable metal devices having favorable mechanical properties and improved ease of binding biologically active agents at a surface thereof; a process for the preparation thereof; the method for their implantation; and the use thereof.

Many orthopaedic implants are presently made of metal. The durability of the bond between an orthopaedic implant and the surrounding tissue is critical to its long term performance. This is influenced by the rate and quality of tissue apposition and, in particular, bleeding complications due to thromboses, and the presence of any infection. Infection is very likely to lead to complications that may require the removal of the implant.

In order to overcome such problems, metal implants have been disclosed with biologically active compounds tethered to the metallic surface by means of a chemical anchor and, optionally, an additional spacer molecule or polymer as disclosed in Wickstrom WO 2005/027990A, Jennisen U.S. Pat. No. 6,635,269 or Dempsey WO 03/008006. Preferred anchors include alkoxysilane reagents that possess terminal chemical functionality, such as 3-aminopropyl triethoxy silane (APTES). It is this functionality that has been used to attach biologically active compounds directly or by means of one or more spacer molecules having just two functional groups for linking at each end. Examples of spacer molecules include aminoethoxyethoxyacetic acid (Wickstrom WO 2005/027990A), glutaric dialdehyde (Jennisen U.S. Pat. No. 6,635,269) or ethylenediamine (Dempsey WO 03/008006). Wickstrom WO 2005/027990A also discloses bi or multifunctional polymer spacers for attaching various biologically active compounds such as antibiotics, thrombolytics, cell growth factors and the like, via amide linkages, ester, methylmaleamide or hydrazone linkages, the disclosure being limited to oligoethylene glycols as bifunctional oligomer spacers and peptides as multifunctional polymer spacers. Dempsey WO 03/008006 also discloses polymer spacers having a plurality of amine groups for attaching proteins such as thrombin inhibitors and growth factors, as biologically active compounds, the disclosure being limited however to polyamine polyethyleneimines (PEI), described as “cross-linking compositions having at least one pendant amino group available for subsequent chemical reaction” and utilizing a further “modifying bifunctional linking molecule” before attachment of the biologically active compound.

The type of reaction required to bond one end of a spacer molecule or polymer to the anchor whilst the other end is used to attach the active, according to Wickstrom, requires quite specific and in many cases complex reactions that can involve multiple deprotection steps. The amount of anchor/linker on the surface is small in comparison to any derivatising solution and so these reactions can easily be affected by the presence of impurities.

There is therefore a need for improved metal implants having acceptable bioactivity and strength, which are capable of being readily implanted with an acceptable success rate, for example without risk of infection leading to implant rejection, or other complication.

In this present invention we provide improved organosilane anchored polymer spacers tethering biologically active agents at a metal surface, but which require only a single process step to anchor the polymer spacer. Moreover there is no need for a bifunctional linking molecule as taught in Dempsey WO 03/008006. In the case of the polymer spacer having multiple target sites, once the organosilane activated metal surface is contacted, the polymers binding is almost certain and so the reaction is less susceptible to the presence of impurities. Similarly there is a better chance that the biologically active agent will bind to the polymer spacer because of the plurality of target sites on the polymer.

Dordick et al, Journal of Nanoscience and Nanotechnology, and related patent application (US 2007/0000781) disclose silicon surfaces or silica nanocolumns for enzyme immobilisation, for conducting biocatalysed reactions or as arrays for sensitive detection devices. Disclosed is attaching APTES (anchor) to a silicon or silica surface and attaching thereto a multifunctional polymer (spacer) which is reactive to amine, and finally attaching thereto an enzyme. The spacer is envisaged as eg poly(ethylene maleic anhydride). There is no disclosure of a modified metal for surface binding active and the modified metal having surface bound active, and the corresponding orthopaedic implant, or of processes for their preparation or of their use in the field of orthopaedic implants and the like.

The present invention relates to improved implantable metal devices comprising an active biosurface; and to an improved process for tethering biologically active agents such as antibiotics, growth factors or biological moderators to a metal surface, in improved manner and with improved reproducibility compared to the known processes.

Accordingly in a first embodiment of a first aspect of the invention there is provided an implantable metal device having an active biosurface, the device comprising a metal surface anchoring a non-peptide polymer spacer having a plurality of target sites by at least one or more of its target sites, a further at least one or more of the target sites binding a biologically active agent, wherein the target sites are derived from reaction of carbonyl, epoxy, hydroxyl or thiol target reactive groups or a combination thereof. Suitably the reaction to anchor polymer spacer or bind biologically active agent is other than by photoreaction. Preferably the target sites are derived from reaction of an excess of carbonyl, epoxy, hydroxyl or thiol target reactive groups or a combination thereof.

In an alternative embodiment of the first aspect of the invention there is provided an implantable metal device having an active biosurface, the device comprising a metal surface anchoring a non-peptide polymer spacer having a plurality of target sites by at least one or more of its target sites, a further at least one or more of the target sites binding a biologically active agent, wherein the biologically active agent is any agent which is capable of modifying the behaviour of cells, particularly microbial cells such as fungal, viral, bacterial, protozoal and the like. In alternative embodiments of the invention the cells are host mammalian cells. Suitably the reaction to anchor polymer spacer or bind biologically active agent is other than by photoreaction.

Preferably the polymer spacer is synthetic. More preferably the polymer spacer is preformed. By this is meant that the polymer spacer is synthetically generated and subsequently anchored to the chemical anchor. This is distinct from a naturally occurring polymer such as cellulose, or a spacer which might be synthetically generated by polymerisation of monomers onto the chemical anchor. Preferably the synthetic polymer spacer is non-oligomeric, more preferably has MW greater than 500 or greater than 1000 as hereinbelow defined or greater than 3000 or has more than 20 or more than 40 or more than 100 repeating units. Preferably the polymer spacer is other than a polyol such as polyethyleneglycol (PEG) or a polyether. Preferably the polymer spacer is substantially non cross-linked, more preferably polymer spacer chains are substantially independent of each other. Preferably attachment at the target sites is achieved by covalent reaction of target reactive groups with the biologically active agent wherein this reaction is other than by photoreaction. This is distinct from a film generated by providing anchor, spacer and reactive group precursors or monomers together with active agent, and reacting in the presence of the metal surface.

Preferably reference hereinbelow to the device is to the device wherein polymer spacer is a synthetic preformed substantially non-crosslinked polymer spacer, and reference to reaction of the polymer spacer is to non photoreactive reaction of the synthetic preformed polymer spacer.

Preferably the device of either embodiment comprises the non-peptide polymer spacer as hereinbefore defined coupled by means of one or more of its target sites to a chemical anchor having one or more anchor sites, wherein the chemical anchor is provided at the metal surface.

In this invention we have discovered multifunctional polymers that can be used as a novel polymer spacer. The anchor is suitably present as a monolayer or polymeric layer at the desired metal surface(s). One or more polymer spacers may be coupled to each chemical anchor. One or more biologically active agents may similarly be coupled to each polymer spacer. Where desired, an excess of target reactive groups of the polymer spacer by means of which both the anchor is bound and the biologically active agent is tethered, ensure effective binding thereof in any desired stoichiometry. An excess may be 2-fold, up to 10-fold, up to 100-fold, up to 1000-fold, up to 10,000-fold or up to 100,000 fold. The use of a polymer spacer also means that the tethered biologically active agent is borne at varying distances from the metal surface thereby maximizing the chance that it will come into contact with target bacterial cells.

In a first embodiment of a second aspect of the invention there is provided a process for generating an active biosurface at the surface of an implantable metal device, comprising contacting a non-peptide polymer spacer having one or a plurality of target reactive groups with a biologically active agent having one or more reactive functional groups and reaction thereof, thereby binding the biologically active agent to the polymer wherein the polymer spacer is anchored to a metal surface of the implantable metal device or subsequently anchoring the polymer spacer to a metal surface of the implantable metal device, wherein target reactive groups are selected from carbonyl, epoxy, hydroxyl, thiol or a combination thereof. Suitably the reaction to anchor polymer spacer or bind biologically active agent is other than by photoreaction. Preferably the polymer spacer has an excess of target reactive groups.

In an alternative embodiment of the second aspect of the invention there is provided a process for generating an active biosurface at the surface of an implantable metal device, comprising contacting a non-peptide polymer spacer having one or a plurality of target reactive groups with a biologically active agent having one or more reactive functional groups and reaction thereof, thereby binding the biologically active agent to the polymer wherein the polymer spacer is anchored to a metal surface of the implantable metal device or subsequently anchoring the polymer spacer to a metal surface of the implantable metal device, wherein the biologically active agent is any agent which is capable of modifying the behaviour of cells, particularly microbial cells such as fungal, viral, bacterial, protozoal and the like. In alternative embodiments of the invention the cells are host mammalian cells. Suitably the reaction to anchor polymer spacer or bind biologically active agent is other than by photoreaction. Preferably the polymer spacer has an excess of target reactive groups.

Preferably the process of either embodiment comprises in a previous or subsequent step anchoring the non-peptide polymer spacer as hereinbefore defined to the metal surface as hereinbefore defined comprising contacting the polymer spacer having one or a plurality of target reactive groups with a chemical anchor having one or more reactive functional groups and reaction thereof, wherein the chemical anchor is provided at the metal surface or is subsequently attached to the metal surface.

Preferably the process comprises in a previous or subsequent step contacting a reactive functional group on the chemical anchor as hereinbefore defined with the metal surface and reaction thereof, thereby attaching the anchored polymer or tethered biologically active agent to the metal surface or generating an activated metal surface having pendant reactive functional groups for anchoring the polymer spacer or the tethered biologically active agent.

In this invention we have discovered a novel process for preparing implantable metal devices having active biosurfaces using multifunctional polymers that can be used as a polymer spacer. After or before attaching a suitable chemical anchor, such as APTES, to a metal surface of the implantable device, one of the anchor's reactive functional groups (amine in the case of APTES) is reacted with a polymer spacer that has a plurality of target reactive groups (such as anhydride where the anchor is APTES). The anchor reacts with and attaches to one or some of these target groups binding the polymer to the anchor and thence to the metal surface. Where desired the target reactive groups on the polymer spacer are present in excess, preferably in large excess, so that many remain unreacted and available for binding, or have previously been reacted with and bound, a biologically active agent in a subsequent or previous reaction step.

The process of the invention presents the following advantages, including improvements over the prior art:
a) reaction conditions are mild, in particular when anchoring the polymer spacer;
b) where desired, an excess of target reactive groups on the polymer spacer means that when an active bearing a reactive functional group such as amine contacts the polymer it is very likely to form a covalent bond;
c) option to maintain an anhydrous or wet environment depending on desired biosurface, preferred choice of anchor etc;
d) there are fewer process steps than the prior art i.e. no multiple deprotection steps;
e) using a preformed polymer spacer further simplifies the process avoiding the need for protection/deprotection and in situ polymerisation reaction, moreover allows use of known MW polymer and may allow control of distribution of target reactive groups.

In a third aspect of the invention there is provided an implantable metal device having a metal surface which is receptive for tethering a biologically active agent, comprising at the metal surface a chemical anchor which anchors a non-peptide polymer spacer having one or a plurality of target reactive groups for tethering a biologically active agent as hereinbefore defined wherein target reactive groups are selected from carbonyl, epoxy, hydroxyl, thiol or a combination thereof. Suitably the reaction to anchor polymer spacer is other than by photoreaction. Preferably the polymer spacer has an excess of target reactive groups.

In a fourth aspect of the invention there is provided a process for the preparation of an implantable metal device having a metal surface which is receptive for tethering a biologically active agent, comprising anchoring a non-peptide polymer spacer having a plurality of target reactive groups wherein the process comprises contacting the target reactive groups of the polymer spacer with a chemical anchor having one or more reactive functional groups and reaction thereof, wherein the chemical anchor is provided at a metal surface of the implantable metal device or is subsequently attached to a metal surface of the implantable metal device, preferably by means of contacting at least one reactive functional group on the chemical anchor as hereinbefore defined with the metal surface and reaction thereof. Suitably the reaction to anchor polymer spacer is other than by photoreaction. Preferably the polymer spacer has an excess of target reactive groups.

In a fifth aspect of the invention there is provided a chemical intermediate comprising an organosilane chemical anchor as hereinbefore defined coupled to one or more target sites of a non-peptide polymer spacer having one or a plurality of target reactive groups for tethering a biologically active agent wherein target reactive groups are selected from carbonyl, epoxy, hydroxyl, thiol or a combination thereof and target sites are derived therefrom. Suitably the reaction to anchor polymer spacer is other than by photoreaction. Preferably the target sites are derived from an excess of target reactive groups.

In a sixth aspect of the invention there is provided a process for the preparation of a chemical intermediate comprising an organosilane chemical anchor as hereinbefore defined coupled to a non-peptide polymer spacer as hereinbefore defined, comprising contacting the polymer spacer having a plurality of target reactive groups with a chemical anchor having one or more reactive functional groups and reaction thereof. Suitably the reaction to anchor polymer spacer is other than by photoreaction. Preferably the target sites are derived from an excess of target reactive groups.

In a first embodiment of a seventh aspect of the invention there is provided a method of treating an animal comprising inserting an implantable metal device comprising an active biosurface into a site in need thereof on said animal, said device comprising a metal surface anchoring a non-peptide polymer spacer by at least one or more of a plurality of target sites, at least a further one or more of the target sites binding a biologically active agent, wherein the target sites are derived from a plurality of carbonyl, epoxy, hydroxyl or thiol target reactive groups or a combination thereof wherein said biologically active agent is capable of interacting with cells adjacent to a surface of the implantable metal device. Suitably the anchoring of polymer spacer or binding of biologically active agent is by reaction other than photoreaction. Preferably the target sites are derived from an excess of target reactive groups.

In a second embodiment of a seventh aspect of the invention there is provided a method of treating an animal comprising inserting an implantable metal device comprising an active biosurface into a site in need thereof on said animal, said device comprising a metal surface anchoring a non-peptide polymer spacer by at least one or more of a plurality of target sites, at least a further one or more of the target sites binding a biologically active agent, wherein the biologically active agent is capable of interacting with and modifying the behaviour of cells, particularly microbial cells such as fungal, viral, bacterial, protozoal and the like. In alternative embodiments of the invention the cells are host mammalian cells. Suitably the anchoring of polymer spacer or binding of biologically active agent is by reaction other than photoreaction. Preferably the polymer spacer comprises an excess of target sites.

In an eighth aspect of the invention there is provided the use of an implantable metal device having an active biosurface as hereinbefore defined as an implantable or subcutaneous metal device, or non-surgical device or a coating thereof, preferably any orthopaedic, cardiovascular, circulatory system or dental implant, tissue engineering device, fixation device, reconstructive device or joint member or trauma-related device or research tool or particles for shaping into such form.

The present invention is applicable to all metallic implant materials. Suitably the metal is selected from Ti and Ti alloys, Zr and Zr alloys, stainless steels, Cr alloys such as Cobalt Chrome, Ta and Ta alloys each of which may be optionally coated, for example with ceramic such as calcium phosphate (hydroxyapatite), zirconium oxide, aluminium oxide or titanium oxide (eg anodised), and the like or a combination thereof.

Preferred metals include pure titanium and titanium alloys such as alloys with chrome, nickel, aluminium, vanadium, cobalt, iron, zirconium, niobium and/or aluminium, preferably titanium/aluminium/vanadium, titanium/aluminium/iron, titanium/zirconium/niobium, titanium/molybdenum/iron (eg Ti-6Al-4V, Ti-5Al-2.5Fe, Ti-13Zr-13Nb, Ti-35Zr-10Nb, Ti-12Mo-6Zr-2Fe), and stainless steels (such as 326L, V2A, V4A, chrome-nickel 316L). Titanium is a metal and alloy constituent which has been used in many biomedical applications and has advantageous bulk and surface properties: a low modulus of elasticity (needed for rigid applications), a high strength to weight ratio (versus stainless steel) and excellent resistance to corrosive environments, due to an oxide layer found on all Ti surfaces.

The metal surface is suitably activated. Preferably the metal surface is oxidized. Oxidation may be by hydrolysis and dehydration, by acid etching, anodisation or passivation. WO 03/008006 discloses processes for removing impure oxidation layers and returning by treatment with deionized water followed by dehydration at elevated temperatures, and acid etching processes. Anodisation and passivation are known in the art. Anodised or passivated metals suitable for the invention are also commercially available.

The implantable metal device may comprise a pure metal or alloy, or may be a combination of a number of different metals, alloys, and the like.

The chemical anchor may be selected from any chemical anchor reported in the literature provided it has a suitable reactive functional group that may be used to anchor the polymer spacer to the surface of the implantable metal device. Suitable chemical anchors comprise organosilanes having at least two reactive functional groups including amino group(s), epoxide(s), hydroxyl, alkoxy, methacrylate(s), epoxy, carboxylic acid(s), chloropropyl group(s), vinyl, vinyl benzyl, mercapto, disulfide, tetrasulfido, anhydride or thiol group(s), or a combination thereof. These are commercially available (Dow Corning). Reference herein to hydroxyl groups is to alcohol groups. Suitably the chemical anchor comprises, two or more reactive groups, preferably two, three or four reactive groups.

Preferably the chemical anchor is selected from organosilanes as hereinbefore defined having at least two reactive functional groups including amino group(s), epoxide(s), hydroxyl, alkoxy, methacrylate(s), epoxy, carboxylic acid(s), chloropropyl group(s), mercapto, disulfide, tetrasulfido, anhydride or thiol group(s), or a combination thereof.

More preferably the chemical anchor is selected from organosilanes including Allylmethyldichlorosilane,

Allyldimethylchlorosilane, Allyltrichlorosilane, Allyltriethoxysilane, Allyltrimethoxysilane,
4-Aminobutyldimethylmethoxysilane,
4-Aminobutyltriethoxysilane,
(Aminoethoxyaminomethyl)phenyltrimethoxyilane,
N-(Aminoethyl)-3-aminopropylmethyldimethoxysilane,
N-(Aminoethyl)-3-aminopropyltrimethoxysilane,
N-(Aminoethyl)-3-aminopropyltriethoxysilane,
N-(6-Aminohexyl)-3-aminopropyl-trimethoxysilane,
3-Aminopropyldimethylethoxysilane,
3-Aminopropyldimethylmethoxysilane (ADMMS),
3-Aminopropylmethyldiethoxysilane,
3-aminopropyltriethoxysilane (APTES),
3-Aminopropyltrimethoxysilane (APTMS),
3-aminopropyldimethylchlorosilane
3-aminopropyldiisopropylethoxysilane
(3,4-Epoxycyclohexyl)ethyltrimethoxysilane,
(3-Glycidoxypropyl)bis(trimethylsiloxy)-methylsilane,
Glycidoxypropyldiisopropylethoxysilane,
3-Glycidoxypropyldimethoxyethoxysilane,
(3-Glycidoxypropyl)methyldiethoxysilane,
3-Glycidoxypropyltrimethoxysilane,
3-Glycidoxypropyltriethoxysilane,
(Mercaptomethyl)dimethylethoxysilane,
(Mercaptomethyl)methyldiethoxysilane,
3-Mercaptopropylmethyldimethoxysilane,
3-Mercaptopropyltrimethoxysilane,
3-Isocyanatopropyltriethoxysilane,
3-carboxypropyltriethoxysilane,
3-(triethoxysilyl)propyl succinic anhydride
N-(hydroxyethyl)-N-methylaminopropyltrimethoxysilane
Bis(2-hydroxyethyl)-3-aminopropyltriethoxysilane, and analogues
   and combinations thereof, optionally with other anchor molecules or surface modifiers. It should be appreciated that this list is not exhaustive and that analogues include mono- di- and tri-alkyl, alkoxy, chloro etc analogues of the above.

More preferably the anchor is selected from 3-aminopropyl triethoxy silane (APTES) or 3-aminopropyl trimethoxy silane (APTMS) or ADMMS or a derivative or analogue thereof, wherein the organic group(s) provide reactive functional group(s) as above defined.

More preferably the anchor is selected from any of the above defined anchors having amine functionality. Most preferably the anchor comprises APTES or APTMS with amine functionality.

Preferably the reaction to anchor the, polymer spacer is a coupling reaction and is not a polymerisation initiated at the anchor molecule.

The reaction coupling the chemical anchor to the metal surface may be performed under anhydrous or wet conditions. Wet conditions suitably comprise reaction in moist organic solvent such as toluene. The reaction is suitably conducted under controlled levels of moisture. Optionally the reaction is conducted in manner to prevent water azeotroping with the solvent.

Preferably the implantable metal comprises the anchor present in an amount sufficient to provide at least a monolayer, and if desired a polymeric anchor layer at the metal surface or such portion of the metal surface which is desired to be bioactive. Should only a portion of the surface be desired to be bioactive, the remainder thereof may be masked or the desired surface portion may be selectively reacted by dipping or painting as known in the art.

Preferably a polymer spacer as hereinbefore defined presents target reactive groups selected from carbonyl, including anhydride, acid chloride, ester, carboxylic acid and the like, epoxy, amine, hydroxyl, thiol and the like, and combinations thereof, with the proviso that a carbonyl group does not comprise ketone. A subset of suitable polymer spacer presents target reactive groups selected from carbonyl, including anhydride, acid chloride, ester, carboxylic acid and the like but excluding ketone, epoxy, hydroxyl, thiol and the like. An alternative subset of suitable polymer spacer presents target reactive groups selected from anhydride, acid chloride, ester, epoxy, amine, hydroxyl, thiol and the like.

Anhydrides include alkyl anhydrides and cyclic anhydrides and the like, and are preferably maleic anhydride, specifically maleic anhydride that is incorporated into the polymer chain as a substituted succinic acid functionality.

For the avoidance of doubt it should be understood that reference herein to maleic anhydride polymer or copolymer is intended as a reference to a polymer or copolymer derived from the polymerisation of maleic anhydride monomer.

Suitably in an implantable metal device according to the invention, the target sites on the polymer spacer are derived from these target reactive groups.

The polymer spacer may be selected from polymers or copolymers of maleic anhydride, alkenes, vinyl monomers including styrene, allylether, and from polymers selected from polyacrylics, polymethacrylics, vinyl polymers, polyamines, polyamides and the like, and compatible combinations thereof.

Copolymers may be random, block, alternating or a random or regular combination thereof.

Preferably a polymer spacer is a copolymer of an anhydride, preferably maleic anhydride, and any monomer with which it will polymerise to provide a multifunctional copolymer.

Preferably a polymer spacer is selected from maleic anhydride isobutylene copolymer (poly MA-alt-iB), maleic anhydride styrene copolymer, maleic anhydride vinyl ether copolymer such as poly MA-alt-methyl vinyl ether, maleic anhydride ethylene copolymer, polymethacryloyl chloride, polyglycidyl methacrylate, polyacrylic acid, polymethacrylic acid, polyvinylamine, polyvinylalcohol, polyvinylalcohol-co-polyvinyl acetate and polyvinylbenzyl thiol and the like, having target reactive groups, or target sites derived therefrom, as defined above. More preferably a polymer spacer is a random, alternating or block copolymer of maleic anhydride, most preferably an alternating or block copolymer of maleic anhydride. Copolymers with isobutylene or vinyl ether are the preferred subset of this group, or with ethylene which are also readily available. These polymers are preferred for an overwhelming number of reasons as follows.

In a particular advantage maleic anhydride polymers with required target reactive groups are readily commercially available. Moreover the anhydride group in the back-bone of the polymer is very reactive towards amines (and to a lesser extent alcohols) requiring only the use of mild reaction conditions on reaction with an anchor molecule at the surface of the implantable metal device. Preferably a plurality of target reactive anhydride groups are distributed throughout the polymer chain providing multiple attachment points (both to the anchor and to the biological active) along the polymer chain, for example the maleic anhydride (MA) copolymers as hereinbefore defined are substantially regular. By virtue of its multiple attachment points, when an amine bearing biologically active agent contacts the polymer it is very likely to form a covalent bond. Finally these polymers are able to bear the biologically active agents at varying distances from the surface of the implantable metal device, maximising the chance that they will come into contact with the target bacterial cells.

In a further advantage these polymers are readily soluble in solvent systems of choice, for example the maleic anhydride copolymers and in particular poly(isobutylene-alt-maleic anhydride) (poly MA-alt-iB) is readily soluble without heating in n-methyl pyrrolidone (NMP) or dimethyl formamide (DMF).

Most preferably the polymer is an anhydride polymer and is reacted with the anchor with subsequent additional heating, causing dehydration and cyclisation resulting in a very strong carbonyl-N-carbonyl bond which is relatively stable under conditions prevailing at implant sites.

Suitably the spacer is preformed as hereinbefore defined and is provided in polymeric form prior to attachment to the anchor. Thereby the process comprises a straightforward attachment step and does not require polymerisation at the anchor surface.

One or a plurality of target sites on the polymer spacer may bind the biologically active agent. Preferably in one embodiment two or more target sites bind the biologically active agent such that it is distributed along the chain length. Preferably in an alternative embodiment one target site binds the biologically active agent to an anhydride polymer spacer as hereinbefore defined.

In the present invention we have discovered that a careful selection of anchor reactive functional groups, polymer spacer and target reactive groups and biologically active agent reactive functional groups enables implantable metal devices to be readily prepared with an active biosurface, without the need for complex or sensitive chemistry. Some combinations of anchor—multifunctional polymer spacer—biologically active agent functionality are given as examples below:

| Anchor/Active | Spacer | |
|---|---|---|
| functionality | functional group | Polymer spacer |
| Amine/hydroxyl | Anhydride | maleic anhydride isobutylene |
| | | alternating copolymer (poly MA- |
| | | alt-iB) |
| | | maleic anhydride styrene |
| | | copolymer (poly MA-styrene) |
| | | maleic anhydride vinyl ether |
| | | alternating copolymer (poly MA- |
| | | alt-ethylene) |
| | | maleic anhydride vinyl ether |
| | | alternating copolymer (poly MA- |
| | | alt-vinyl ether) |
| | acid chloride | polymethacryloyl chloride |
| | epoxy | polyglycidyl methacrylate |
| | carboxylic acid | polyacrylic acid |
| | | polymethacrylic acid |
| methacrylate | Amine | polyvinylamine |
| epoxy | amine | polyvinylamine |
| | hydroxyl | polyvinylalcohol |
| | | polyvinylalcohol-co-polyvinyl |
| | | acetate |
| Chloropropyl | Amine | Polyvinylamine |
| vinyl, vinyl | Amine | Polyvinylamine |
| benzyl | | |
| mercapto, | Thiol | polyvinylbenzyl thiol |
| disulfide, | | |
| tetrasulfido | | |
| Anhydride | amine | polyvinylamine |
| | hydroxyl | polyvinylalcohol |
| | | polyvinylalcohol-co-polyvinyl |
| | | acetate |

Preferably the process as hereinbefore defined is performed in solvent. Solvent may be selected from any suitable solvents for the entities to be reacted and in particular any organic solvent capable of solubilising the polymer. In a particular advantage we have found that DMF or NMP may be used in reactions with the polymer spacer.

The anchor may be attached to the surface by covalent or non-covalent eg van der Waals, ionic or hydrophobic links or the like. Similarly the biologically active agent may be attached to the spacer by covalent or non-covalent eg van der Waals, ionic or hydrophobic links or the like. The biologically active agent may be linked via PEG or an acid-labile linkage. Alternatively, the polymer to biologically active agent bond may be direct, without any intermediate moiety, in particular without a bifunctional linking molecule.

Suitably the polymer spacer has molecular weight of sufficient order to bear multifunctionality, and in particular to bear target reactive groups in excess for the envisaged reactions. Preferably the polymer spacer is characterised by molecular weight in excess of 500 (Mw), more preferably in the range 500 to 2×10⁶, preferably 1,000 to 2×10⁶, more preferably in excess of 4,000 to 1×10⁶, more preferably from 6000 to 1×10^{6,} more preferably from 6000 to 1×10⁵, for example in excess of 10,000 to 1×10⁵. Higher molecular weight polymers present the possibility to bind a plurality of biologically active agent molecules. Polymers in weight range Mw 4,000, 6,000 or 10,000 up to 1×10⁵ or 6×10⁴ are expected to provide this advantage and yet be of sufficiently low molecular weight to be soluble in readily available solvents.

The biologically active agent may comprise any reactive functional group enabling it to react with and be tethered by the polymer spacer as hereinbefore defined. Preferably the biologically active agent contains one or more amino, carboxylic acid, hydroxyl or thiol reactive functional groups or salts thereof. Suitable salt forms are known in the art and include hydrochloride, sulphate and the like.

The biologically active agent may be selected from a growth factor, an antimicrobial, such as an antibacterial, an agent which inhibits the deposition of a bacterial biofilm onto the surface of the implanted metal device, or an antibiotic, or other agent able to modify the behaviour of bacteria, a bone morphogenetic factor, a chemotherapeutic agent, a pain killer, a bisphosphonate, a bone growth agent, an angiogenic factor, an adhesion peptide or other biological moderator to a metal surface and combinations thereof.

A growth factor may be selected from the group consisting of platelet derived growth factor (PDGF), VEGF, ECGF, transforming growth factor b (TGF-b), insulin-related growth factor-I (IGF-I), insulin-related growth factor-II (IGF-II), fibroblast growth factor (FGF), beta-2-microglobulin (BDGF II), bone morphogenetic protein (BMP), and combinations thereof.

Suitable biologically active agents in this embodiment of the invention include anti-microbials, in particular bactericides and agents which prevent adhesion of bacteria or are able to penetrate the bacterial cell wall and affect behaviour, such as antibiotics and antibacterial compounds, biofilm inhibitors and the like.

This method is suitable for antibiotics and combinations thereof, such as aminoglycosides, cephalosporins, glycopeptides, macrolides, quinolones, penicillins, tetracycline hydrochloride, glycylcyclines such as minocycline, tigecyclin etc. that contain a suitably reactive functional group such as amino, carboxylic acid, hydroxyl or thiol.

Representative examples grouped by reactive functional group are:

### a) Amine

Vancomycin (glycopeptide)
Gentamycin (aminoglycoside)
Tobramycin (aminoglycoside)
   b) Carboxylic acid
Cefazolin (cephalosporin)
Vancomycin (glycopeptide)
Flucloxacillin (penicillin)

### c) Hydroxyl

Vancomycin (glycopeptides)
Erythromycin (macrolide)
and the like.

Antibiotics that contain a primary amine are preferred.

A factor may also be selected from the group consisting of proteins of demineralized bone, demineralized bone matrix (DBM), bone protein (BP), bone morphogenetic protein (BMP), osteonectin, osteocalcin, osteogenin, and combinations thereof.

Preferably the biologically active agent is an agent which is able to penetrate the bacterial cell wall and affect behaviour, for example an antibiotic or the like as hereinbefore defined, optionally in combination with one or more other agents for treatment of conditions such as cancer, restenosis, bone loss, thromboses and the like as known in the art. Preferably an antibiotic is vancomycin optionally in combination with one or more other agents.

The biologically active agent may have a labile or non-labile bond to the polymer. An acid or enzyme labile bound biologically active agent may be released at the implant site to create a surrounding biologically active zone. Alternatively the biologically active agent may remain bound to the implantable metal device and be non labile in the presence of acid or enzyme whereby the implant retains biological activity.

Vancomycin is a therapeutic molecule that may be tethered to the surface of an implantable metal device of the invention, using a flexible linkage that may not be cleaved and yet still places the vancomycin within the cell. Vancomycin inhibits bacterial cell wall synthesis by inhibiting peptidoglycan synthesis, a process that occurs on the exterior surface of the cell membrane. Membrane anchored vancomycin would be expected to display an increased activity against resistant organisms. Immobilised vancomycin on a flexible, non-labile linker, could be bactericidal without release from the metal surface. The presence of the polymer spacer can allow passage through the bacterial cell wall while still maintaining contact with the metal surface. This allows the cell to be killed but remain in the implant zone, and the vancomycin remains in place to attack further bacterial cells. In a further advantage by not releasing the vancomycin, there is a reduced risk that the antibiotic can break away and cause resistance.

In a further aspect of the invention there is provided a method of treating an animal as hereinbefore defined wherein the biologically active agent provided by the implantable metal device is capable of interacting with cells adjacent to a surface of the implantable metal device. Suitably the agent is capable of interacting with cells which are adjacent to the device and able to deleteriously affect the device or the animal by interacting with the device. In a particular advantage of the invention the polymer spacer tethers the biologically active agent in manner that it is able to interact with cells in a zone surrounding the surface of the device, suitably in a zone of diameter equivalent to the distribution of target reactive groups throughout the polymer.

An animal may include mammals, in particular humans or other animals.

The cells may be any cells with which it is desired that the biologically active agent interacts. Preferably in the method the cells are microbial cells, including fungal, viral, bacterial, protozoal or the like. Preferably the biologically active agent is an antimicrobial, more preferably the cells are bacterial cells and the biologically active agent is selected from an an antibacterial, an agent which inhibits the deposition of a bacterial biofilm onto the surface of the implanted metal device, or an antibiotic. A suitable biologically active agent is as hereinbefore defined, and is preferably an antibiotic comprising vancomycin.

Suitably bacterial cells with which the biologically active agent is able to interact are gram positive, preferably are at least one of a Staphylococcus, Streptococcus, Bacillus species or gram positive anaerobes, more preferably a Staphylococcus spp. is S. aureaus or S. epidermis.

In further embodiments of the invention the cells are host mammalian cells.

An implantable metal device of the invention may be in the form of, or suitable for shaping in the form of any implantable or subcutaneous metallic devices, or non-surgical devices or a coating thereof, preferably any orthopaedic, cardiovascular, circulatory system or dental implant, tissue engineering device, fixation device, reconstructive device or joint member or trauma-related device or research tool or particles for shaping into such form.

Examples of orthopaedic implants include intermedullary nails, interference screws, fracture fixation plates, pins, external fixator pins, wires and reconstructive devices or joint members including hip, knee, elbow and shoulder joint replacements, preformed bone replacements and bone filler.

Examples of cardiovascular implants include stents, pacemakers, artificial organs such as total implantable heart and ventricular assist devices, valves such as mechanical heart valves, valve housing chambers, access ports, ports for hemodialysis and surgical clips.

Examples of circulatory system implants include catheters, needles and the like.

Examples of dental implants include dental posts.

Examples of tissue engineering devices include porous and non-porous scaffolds, plates such as maxillo-facial plates or fracture fixation plates, rods, fibres, bone graft substitutes or fillers, implants and the like.

Examples of fixation devices include suture anchors, soft tissue anchors, screws such as bone or interference screws, pins, plates, rods, nails, spikes and staples, mesh for spinal infusion and external fixators.

Examples of trauma-related devices include fixation devices or spinal implants.

Examples of non-surgical devices include research tools for culturing an implantable device in vitro and the like.

The implantable device may be in the form of a standard or custom shaped device or may be in the form of particles which may be located in situ to fill a desired space. Particles may take the form of a jack, a tablet, a strip, a block, a cube, a chip, a pellet, a pill, a lozenge, a sphere, a ring, gel, putty, paste, formable granules, or powder and combinations thereof. Preferably particles take the shape of a jack which is a 4, 5 or 6 arm star shape, and more preferably a particle is a JAX™ bone void filler.

Particles are suitably of the order of 0.1 to 2 cm in greatest dimension, preferably 0.1 to 1.25 cm, depending on the intended use, more preferably less than about 1 cm in diameter, most preferably in the range of 0.2 to 1 cm.

Preferably an implantable metal device as hereinbefore defined is shaped by machining, forging, casting or moulding a metal to the desired shape, as known in the art. Alternatively an implantable metal device may be shaped by obtaining a plurality of metal particles as hereinbefore defined, and shaping or packing to desired shape, followed by treatment with an element of the invention.

An implantable metal device as hereinbefore defined may be suitable for cosmetic or non-cosmetic therapeutic purpose which may include research, surgery, dental applications and the like.

The present invention is now illustrated in non-limiting manner by reference to the following examples.

### EXAMPLE 1

### Preparation of Implantable Device Substrate

Ti6Al4V 0.25 inch×0.25 inch diameter cylindrical stubs (commercially available) were obtained having a surface rich in oxide and/or hydroxide groups as follows: the stubs were degreased and cleaned by washing in absolute alcohol to remove any residual processing oils etc, and then air dried. The stubs were sonicated in hydrochloric acid, washed and dried in an oven overnight at 160° C.

### EXAMPLE 2

### Introducing the Organosilane Anchor

The cleaned stubs may be reacted with APTES according to methods described in U.S. Pat. No. 6,635,269 (Jennisen) as follows: 0.5 g of titanium stubs are added to 9 ml distilled water and 0.2-2 ml of 3-aminopropyltriethylethoxysilane are added and adjusted to pH 3-4. After regulation of pH the reaction solution is incubated in a water bath for 2h at 75° C. The metal stubs are optionally washed in a solution of mild aqueous base. Subsequently the activated metal is separated from the reaction mixture and is dried in a drying cabinet at 115° C.

### EXAMPLE 3

### Introducing the Multifunctional Polymer Spacer

The functionalized dried stubs were reacted with a solution of maleic anhydride isobutylene alternating copolymer (poly MA-alt-iB, approximately 6000 Mw) in solvent (NMP, DMF or other solvent capable of solubilising the polymer), e.g. in 5% w/v NMP for 1 hour.

### EXAMPLE 4

### Introducing the Biological Agent

The polymer-bearing surface was cleaned (3×10 ml NMP) and functionalised with vancomycin by treating with a 1.8% wt/vol solution of vancomycin in solvent.

### EXAMPLE 5

### Detection of Bound Vancomycin

a) Vancomycin was detected on titanium stubs by immuno-fluorescence staining as known in the art.

b) Elution in phosphate buffered saline over three days showed small amounts (ng's) (consistent with residual amounts) vancomycin released but thereafter no further release detectable up to a week.

### EXAMPLE 6

### Alternative Reaction Condition with Silane

A variety of reaction conditions can be used to attach APTES to metallic surface that include both wet and anhydrous conditions.

Anodised, toluene-washed 0.25 inch×0.25 inch cylindrical Ti6Al4V alloy stubs were heated in a solution of 10 v/v % APTES in dry toluene at reflux for 4 hours. The toluene was repeatedly condensed through a Soxhlet extractor filled with type 13X sieves to remove ethanol bi-product. The stubs were drained, washed in dry toluene, air-dried and then placed in an oven at 110° C. for 30 mins.
The stubs were treated with 2-methoxy-2,4-diphenyl-3(2H) furanone solution (MDPF) a fluorescent stain by placing them in a 50:50 mixture of a 1 mg/ml solution of MDPF in acetonitrile and 50 mM borate buffer at pH 9 then left to stand overnight. This dye attaches to the amine group on APTES.
On the following day the samples were washed three times with PBS and then viewed using a Leica model DMLB microscope.
Untreated samples showed no fluorescence whilst the APTES showed a blue fluorescence over the surface. This indicated that surface functionalisation by the silane had been successful.

### EXAMPLE 7

### Alternative Anchor

An alternative anchor, with a single anchor point i.e. aminopropyldimethylmethoxysilane (ADMMS) was used as an alternative to APTES.

Toluene washed 0.25 inch×0.25 cylindrical Ti6Al4V alloy stubs were left overnight in de-ionised water. These were added to dried toluene (100 ml) followed by ADMMS (5 ml) and de-ionised water (2 g). The vessel was equipped with a Dean and Stark still head. The mixture was brought to reflux and refluxed for 4 hours. Over the reflux period any water that azeotroped with the toluene and was removed. The stubs were removed from the reaction mixture and washed in dried toluene, air-dried and then heated in an oven at 110° C. for 30 mins.
The ADMMS functionalised stubs were warmed in an oven for 30 mins then added to a solution of poly(isobutylene-alt-maleic anhydride) {MA-alt-iB polymer} approximate molecular weight 6,000 in N-methylpyrrolidone (NMP) (1% w/v). The mixture was warmed and allowed to react for 1 hour. The stubs were removed and washed in anhydrous NMP. These stubs were transferred to a solution of vancomycin hydrochloride (1% w/v) in dried N,N-dimethylformamide/triethylamine (99/1 v/v) and left at ambient temperature overnight. The stubs were washed in de-ionised water then in tetrahydrofuran and finally air-dried
Immuno-histochemical staining for vancomycin produced low levels of green fluorescence showing that vancomycin had been successfully tethered to the sample surfaces.

### EXAMPLE 8

### Other Metals

Other metals that have an oxide layer are suitable. Stainless steel is a common implant material. This example covers the functionalisation of stainless steel.

Passivated stainless steel k-wires (0.8 mm diameter×70 mm long) were treated in a similar manner to example 6 to attach APTES by reacting with 5 v/v % APTES under dry conditions at elevated temperature below reflux to obtain surfaces rich in amine groups.
The APTES functionalised wires were warmed in an oven to dry the wires for 30 minutes then added directly to a solution of MA-alt-iB polymer (1% w/v) in anhydrous NMP. The solution was warmed and allowed to react for 1 hour. The pieces were washed in anhydrous NMP then added directly to a solution of vancomycin hydrochloride (1% w/v) in dried N,N-dimethylformamide (DMF). Dried triethylamine (1% v/v) was added with agitation. After leaving at ambient temperature overnight the test pieces were washed thoroughly in de-ionised water then tetrahydrofuran (THF) and allowed to dry.
Immuno-histochemical staining for vancomycin produced a green fluorescence indicating that vancomycin had been tethered to the surface.

### EXAMPLE 9

### Alternative Molecular Weight Linker

This example shows that a range of molecular weights of polymer can be used.

APTES functionalised cylindrical machine finish titanium Ti6Al4V 0.25 inch×0.25 inch stubs produced as described in EXAMPLE 6 were reacted separately with MA-alt-iB polymer of approximate weight average molecular weights 6,000 and 60,000 using the conditions summarized in Table 1. The 60,000 molecular weight polymer was not substantially soluble in NMP and so a different solvent, N,N-dimethylformamide (DMF), was used.

TABLE 1
attachment conditions

| | MA-alt-iB polymer | MA-alt-iB polymer |
|---|---|---|
| | (Ex: Aldrich) | (Ex: Aldrich) |
| | Typical M_{w} = 6,000 | Typical M_{W} = 60,000 |
| Solvent | NMP | DMF |

| | |
|---|---|
| Polymer | 1% w/v |
| concentration | |
| Reaction time | 1 h |

| | | |
|---|---|---|
| Post reaction | NMP wash | DMF wash |

| | |
|---|---|
| Vancomycin | 1% w/v Vancomycin hydrochloride in dry 1% |
| Attachment | v/v triethylamine in DMF, ambient |
| | temperature overnight |
| Post reaction | Exhaustive wash in de-ionised |
| | water/tetrahydrofuran rinse/air-dry |

In each case control samples with polymer alone were isolated, washed in tetrahydrofuran and allowed to dry.
Immuno-histochemical staining showed no fluorescence on the control stubs with the polymer alone. For both polymers, stubs reacted with vancomycin showed green fluorescence indicating that the tethering had been successful.

### EXAMPLE 10

### Alternative Maleic Anhydride Polymer Linkers

An important aspect of the invention is that the polymer contains maleic anhydride residues. Several other alternating copolymers are available. These were evaluated as materials to bind vancomycin.

The following polymers, all ex Aldrich, were used: poly(methyl vinyl ether-alt-maleic anhydride) typical M_{w}=216,000, poly(ethylene-alt-maleic anhydride) typical M_{w}=100,000 to 500,000, and poly(isobutylene-alt-maleic anhydride) typical M_{w}=6,000. For each polymer, 5 stubs pre-functionalised with APTES were warmed in an oven for 30 mins then added to a 1 w/v % solution of polymer in anhydrous N-methylpyrrolidone (NMP). In each case the polymer was dissolved in solvent by stirring at room temperature. The mixture was warmed and the reaction allowed to proceed for 1 hour. After washing in fresh anhydrous NMP, 3 of the stubs were added to a solution of 1% w/v vancomycin hydrochloride in dried triethylamine/N,N-dimethylformamide (1:99 v/v) and left overnight at room temperature. The remaining two stubs, controls with polymer alone) were washed in tetrahydrofuran and allowed to air-dry.
Immuno-histochemical staining for vancomycin produced surfaces with green fluorescence for all polymers showing that vancomycin had been successfully tethered to the sample surfaces

### EXAMPLE 11

### Alternative Antibiotics

The technology is also capable of binding other antibiotics. Here gentamicin was bound.

APTES functionalised cylindrical titanium Ti6Al4V 0.25 inch×0.25 inch stubs produced were treated with MA-alt-iB polymer of approximate molecular weight 6 000. The stubs were heated for 1 hour in the presence of a 1 w/v % solution of the polymer in anhydrous N-methylpyrrolidone (NMP). The stubs were washed in fresh NMP and then treated overnight with a suspension of 1 w/v % gentamicin sulphate in dried triethylamine/anhydrous NMP (1/99 v/v) before extensive extraction in water. The stubs were rinsed in tetrahydrofuran and air-dried.
The stubs were evaluated using immuno-histochemical staining for gentamicin as known in the art. A goat polyclonal IgG anti-gentamycin primary antibody (AbD Serotec) was used followed by an Alexa Fluor 488 donkey anti-goat IgG secondary antibody (Invitrogen). The Alexa Fluor tag on the secondary antibody fluoresces green. Stubs with gentamicin exhibited green fluorescence whereas stubs with APTES and poly(isobutylene-alt-maleic anhydride) functionalised stub controls did not. This indicated that gentamicin had been successfully linked onto the surface.

### EXAMPLE 12

### Detection of Bound Vancomycin

Grit blast finish titanium Ti6Al4V stubs with attached vancomycin were prepared according to EXAMPLES 6 (attachment of APTES) and 9 (Using MA-alt-iB polymer of typical M_{w} 6,000) and were assessed against control uncoated stubs using live/dead-staining after culture in a flow cell.

Inoculated broth (Tryptone Soya Broth or TSB) was prepared to a concentration of 10⁴ cfu/ml using an S. aureus suspension, prepared in Maximum Recovery Diluent (MRD) from an 18 hour broth culture, grown at 37° C. The inoculated broth was pumped through flow cells containing the stubs at 1 ml per minute for 3 hours. The cells were flushed with fresh sterile TSB at a rate of 0.5 ml per minute for 18 hours.
Following incubation the stubs were transferred to wells in a sterile 24 well plate. Samples were rinsed in PBS and the excess PBS removed using a vacuum pump. This washing was repeated 5 further times (i.e. 6 in total) for each sample. Stubs were then transferred to wells in a 48 well plate.
Washed stubs were stained for 20 mins using 0.6 ml of 1× Baclight Live/Dead stain in individual wells of a 48 well plate wrapped with foil. Stubs were then washed a further 3 times with PBS as above. The stubs were imaged using a Leica DMRE upright microscope with TCS-SP confocal.
Overall, in terms of blind scoring, surfaces with bound vancomycin showed reduced colonisation by biofilm when compared with their equivalent uncoated control samples.

1. An implantable metal device having an active biosurface, the device comprising a metal surface anchoring a non-peptide polymer spacer having a plurality of target sites, by at least one or more of its target sites, a further at least one or more of the target sites binding a biologically active agent, wherein the target sites are derived from reaction of a plurality of carbonyl, epoxy, hydroxyl or thiol target reactive groups or a combination thereof wherein the reaction to anchor polymer spacer or bind biologically active agent is other than by photoreaction.

2. An implantable metal device having an active biosurface, the device comprising a metal surface anchoring a non-peptide polymer spacer having a plurality of target sites by at least one or more of its target sites, a further at least one or more of the target sites binding a biologically active agent, wherein the biologically active agent is any agent which is capable of modifying the behaviour of cells and wherein the reaction to anchor polymer spacer or bind biologically active agent is other than by photoreaction.

3. An implantable metal device as claimed in claim 1 wherein the non-peptide polymer spacer as hereinbefore defined is coupled by means of one or more of its target sites to a chemical anchor having one or more anchor sites, wherein the chemical anchor is provided at the metal surface.

4. A device as claimed in claim 1 wherein target sites are rederived from reaction of an excess of target reactive groups.

5. A device as claimed in claim 1 wherein a chemical anchor comprises an organosilane having at least two reactive functional groups including amino group(s), epoxide(s), hydroxyl, alkoxy, methacrylate(s), epoxy, carboxylic acid(s), chloropropyl group(s), mercapto, disulfide, tetrasulfido, anhydride or thiol group(s), or a combination thereof.

6. A device as claimed in claim 1 wherein the target sites are derived from target reactive groups selected from carbonyl, including anhydride, acid chloride, ester, carboxylic acid and the like, epoxy, amine, hydroxyl, thiol and the like, and combinations thereof.

7. A device as claimed claim 1 wherein the polymer spacer is selected from polymers or copolymers of maleic anhydride, alkenes, vinyl monomers including styrene, allylether, polyols including glycols, and ethers, and from polymers selected from polyacrylics, polymethacrylics, vinyl polymers, polyamines, polyamides and the like, and compatible combinations thereof.

8. A device as claimed in claim 1 wherein a polymer spacer is selected from maleic anhydride isobutylene copolymer, maleic anhydride styrene copolymer, maleic anhydride vinyl ether copolymer, maleic anhydride ethylene copolymer, polymethacryloyl chloride, polyglycidyl methacrylate, polyacrylic acid, polymethacrylic acid, acrylic acid methacrylic acid copolymer, polyvinylamine, polyvinylalcohol, polyvinylalcohol-co-polyvinyl acetate and polyvinylbenzyl thiol and the like, having reactive functional groups or polymer to anchor bond derived therefrom as defined above.

9. A process or device as claimed in claim 1, wherein a polymer spacer is an alternating or block copolymer of maleic anhydride.

10. A device as claimed in claim 1, wherein the polymer to biologically active agent bond is direct, without any intermediate moiety, in particular without a bifunctional linking molecule.

11. A device as claimed in claim 1, wherein a biologically active agent is selected from a growth factor, an antimicrobial, such as an antibacterial, an agent which inhibits the deposition of a bacterial biofilm onto the surface of the implantable metal device, or an antibiotic, or other agent able to modify the behaviour of bacteria, a bone morphogenetic factor, a chemotherapeutic agent, a pain killer, a bisphosphonate, a bone growth agent, an angiogenic factor, an adhesion peptide or other biological moderator to a metal surface and combinations thereof.

12. A device as claimed in claim 1, wherein an implantable metal device is in the form of, or suitable for shaping in the form of any implantable or subcutaneous metallic devices, or non-surgical devices or a coating thereof, preferably any orthopaedic, cardiovascular, circulatory system or dental implant, tissue engineering device, fixation device, reconstructive device or joint member or trauma-related device or research tool or particles for shaping into such form.

13. A process for generating an active biosurface at the surface of an implantable metal device, comprising contacting a non-peptide polymer spacer having one or a plurality of target reactive groups with a biologically active agent having one or more reactive functional groups and reaction thereof, thereby binding the biologically active agent to the polymer wherein the polymer spacer is anchored to a metal surface of the implantable metal device or subsequently anchoring the polymer spacer to a metal surface of the implantable metal device, wherein target reactive groups are selected from carbonyl, epoxy, hydroxyl, thiol or a combination thereof, and wherein the reaction to anchor polymer spacer or bind biologically active agent is other than by photoreaction.

14. A process for generating an active biosurface at the surface of an implantable metal device, comprising contacting a non-peptide polymer spacer having one or a plurality of target reactive groups with a biologically active agent having one or more reactive functional groups and reaction thereof, thereby binding the biologically active agent to the polymer wherein the polymer spacer is anchored to a metal surface of the implantable metal device or subsequently anchoring the polymer spacer to a metal surface of the implantable metal device, wherein the biologically active agent is any agent which is capable of modifying the behaviour of cells and wherein the reaction to anchor polymer spacer or bind bilologically active agent is other than by photoreaction.

15. A process as claimed in claim 13 which comprises in a previous or subsequent step anchoring the non-peptide polymer spacer to the metal surface comprising contacting the polyrner spacer having one or a plurality of target reactive groups with a chemical anchor having one or more reactive functional groups and reaction thereof, wherein the chemical anchor is provided at the metal surface or is subsequently attached to the metal surface.

16. A process as claimed in claim 13 which comprises in a previous or subsequent step contacting at least one reactive functional group on the chemical anchor with the metal surface and reaction thereof, thereby attaching the anchored polymer or tethered biologically active agent to the metal surface or generating an activated metal surface having pendant reactive functional groups for anchoring the polymer spacer or the tethered biologically active agent.

17. A process as claimed in claim 13 wherein any of the metal, chemical anchor, polymer spacer, biologically active agent, configuration or shape thereof is as defined in any of claims 4 to 12.

18. An implantable metal device having a metal surface which is receptive for tethering a biologically active agent to form an implantable metal device having an active biosurface, comprising a chemical anchor at the surface which anchors a non-peptide polymer spacer having one or a plurality of target reactive groups for tethering a biologically active agent as hereinbefore defined wherein target reactive groups are selected from carbonyl, epoxy, hydroxyl, thiol or a combination thereof and wherein the reaction to anchor polymer spacer is other than by photoreaction.

19. A process for the preparation of an implantable metal device having a metal surface which is receptive for tethering a biologically active agent as claimed in claim 18, comprising anchoring a non-peptide polymer spacer having one or a plurality of target reactive groups wherein the process comprises contacting the target reactive groups of the polymer spacer with a chemical anchor having one or more reactive functional groups and reaction thereof, wherein the chemical anchor is provided at a metal surface of the implantable metal device or is subsequently attached to a metal surface of the implantable metal device, preferably by means of contacting at least one reactive functional group on the chemical anchor as hereinbefore defined with the metal surface and reaction thereof.

20. A chemical intermediate comprising an organosilane chemical anchor as hereinbefore defined coupled to one or more target sites of a non-peptide polymer spacer having one or a plurality of target reactive groups for tethering a biologically active agent wherein target reactive groups are selected from carbonyl, epoxy, hydroxyl. thiol or a combination thereof and target sites are derived from reaction thereof, wherein the reaction to anchor polymer spacer is other than by photoreaction.

21. A process for the preparation of a chemical intermediate as claimed in claim 20, comprising an organosilane chemical anchor coupled to a non-peptide polymer spacer as hereinbefore defined in claim 20, comprising contacting the polymer spacer having a plurality of target reactive groups with a chemical anchor having one or more reactive functional groups and reaction thereof wherein the reaction to anchor polymer spacer is other than by photoreaction.

22. A method of treating an animal comprising inserting an implantable metal device comprising an active biosurface into a site in need thereof on said animal, said device comprising a metal surface anchoring a non-peptide polymer spacer by at least one or more of a plurality of target sites, at least a further one or more of the target sites binding a biologically active agent, wherein the target sites are derived from reaction of a plurality of carbonyl, epoxy, hydroxyl or thiol target reactive groups or a combination thereof wherein the reaction to anchor polymer spacer or bind biologically active agent is other than by photoreaction wherein said biologically active agent is capable of interacting with cells, adjacent to a surface of the implantable metal device.

23. A method of treating an animal comprising inserting an implantable metal device comprising an active biosurface into a site in need thereof on said animal, said device comprising a metal surface anchoring a non-peptide polymer spacer by at least one or more of a plurality of target sites, at least a further one or more of the target sites binding a biologically active agent, wherein the biologically active agent is capable of interacting with and modifying the behaviour of cells and wherein the reaction to anchor polymer spacer or bind biologically active agent is other than by photoreaction.

24. A method according to claim 22, wherein the cells are microbial cells and the biologically active agent is an antimicrobial.

25. A method according to claim 24, wherein the cells are bacterial cells and the biologically active agent is selected from an antibacterial, an agent which inhibits the deposition of a bacterial biofilm onto the surface of the implant, or an antibiotic.

26. A method according to claim 22, wherein the biologically active agent contains an amino, carboxylic acid, hydroxyl or thiol reactive functional group or salt thereof.

27. A method according to claim 25, wherein the antibiotic is vancomycin.

28. A method according to claim 25, wherein the bacteria is gram positive.

29. A method according to claim 28, wherein the gram positive bacteria is at least one of a Staphylococcus, Streptococcus, Bacillus species or gram positive anaerobes.

30. A method according to claim 28, wherein the Staphylococcus spp. is S. aureaus or S. epidermis.

31-32. (canceled)
